(19) United States
(12) Patent Application Publication
(10) Pub. No.: US 2004/0101903 A1
(43) Pub. Date: May 27, 2004

(54) Method and apparatus for sequence annotation

(75) Inventor: Isidore Rigoutsos, Astoria, NY (US)

Correspondence Address: Ryan, Mason & Lewis, LLP, Suite 205, 1300 Post Road, Fairfield, CT, 06430, US

(73) Assignee: International Business Machines Corporation, Armonk, NY (US)

(21) Appl. No.: 10/305,582

(22) Filed: Nov. 27, 2002

(51) Int. Cl. G06F 7/00; G06F 17/30; G01N 33/53; G06F 19/00; G01N 33/48; G01N 33/50

(52) U.S. Cl. 435/7.1; 702/19

(57) ABSTRACT

Techniques for annotating sequences. In one aspect of the invention, a method is provided for annotating a query sequence. The method comprises the following steps. Patterns associated with a database, comprising annotated sequences, are accessed. Attributes are assigned to the patterns based on the annotated sequences. The patterns with assigned attributes are used to analyze the query sequence.

### FIELD OF THE INVENTION

The present invention relates to sequence analysis and, more particularly, to the annotation of sequences.

### BACKGROUND OF THE INVENTION

Research efforts have long been focused on the search for computational methods to determine the properties of a protein, including functional, structural and physiochemical properties, directly from the corresponding amino acid sequence. The number of amino acid sequences that are being deposited in public databases has been increasing steadily with the advances in sequencing methods and systems. The elucidation of the properties of a protein using the sequences in these databases typically involves tedious manual analysis. As thousands of previously unknown proteins, as well as increasing numbers of complete genomes, are being made publicly available, less labor intensive methods for protein analysis are sought. Protein annotation, importantly, is the first step in the attempt to fully describe a particular organism through characterization of its metabolic pathways and transcription regulation networks.

As such, a demand exists for an automated approach to annotate individual sequences, as well as complete genomes, quickly, exhaustively and objectively.

### SUMMARY OF THE INVENTION

The present invention provides techniques for annotating sequences. In one aspect of the invention, a method is provided for annotating a query sequence. The method comprises the following steps. Patterns associated with a database, comprising annotated sequences, are accessed. Attributes are assigned to the patterns based on the annotated sequences. The patterns with assigned attributes are used to analyze the query sequence.

The patterns with assigned attributes may be used to define an attribute vector, the attribute vector characterizing portions of the query sequence. The patterns with assigned attributes may be stored in a database. The query sequence may be a polypeptide sequence comprising amino acids. The attribute vector may comprise a number of counters, wherein the number of counters is proportional to the number of amino acid residues in the query sequence. The assigned attributes may be used to contribute values to counters of the attribute vector that correspond to portions of the query sequence matched by the corresponding patterns. Further, a score may be determined for the patterns with assigned attributes used to define the attribute vector, wherein the score represents a degree of similarity between the query sequence and the annotated sequences of the database.

A more complete understanding of the present invention, as well as further features and advantages of the present invention, will be obtained by reference to the following detailed description and drawings.

### BRIEF DESCRIPTION OF THE DRAWINGS

FIG. 1 is a flow chart illustrating an exemplary methodology for annotating a query sequence according to an embodiment of the present invention;

FIG. 2 is a block diagram of an exemplary hardware implementation of a method for annotating a query sequence according to an embodiment of the present invention;

FIG. 3 is a flow chart illustrating an alternate exemplary methodology for annotating a query sequence according to an embodiment of the present invention;

FIG. 4 is a schematic diagram illustrating an exemplary implementation according to an embodiment of the present invention;

FIGS. 5(A) through 5(I) are plots showing some of the results of the annotation of human ubiquitin according to an embodiment of the present invention;

FIGS. 6(A) through 6(D) are plots showing some of the results of the annotation of the sequence VVVTAHAF according to an embodiment of the present invention; and

FIGS. 7(A) through 7(B) are plots showing some of the results of the annotation of the adrenocorticotropic hormone receptor protein according to an embodiment of the present invention.

### DETAILED DESCRIPTION OF PREFERRED EMBODIMENTS

The present invention will be described below in the context of an illustrative protein sequence annotation methodology. However, it is to be understood that the present invention is not limited to such a particular protein sequence annotation methodology. Rather, the invention is more generally applicable to any sequence annotation, as would be apparent to a person of ordinary skill in the art. Thus, the teachings of the present invention should not be construed as being limited to the analysis of a protein sequence. As such, the teachings of the present invention are more generally applicable to the annotation of sequences.

Automated elucidation of the properties of a protein directly from an amino acid sequence, as described herein, is beneficial as it minimizes the amount of manual labor associated with the annotation process. The automated elucidation process typically proceeds by accessing repositories of previously accumulated knowledge and using computation, i.e., in silico approaches, to replace generally tedious manual analysis. The discovery of protein properties directly from the corresponding amino acid sequence, in an automated or semi-automated manner, is an important goal as the information on thousands of previously unknown proteins is now being made publicly available.

Numerous methods have been proposed for determining protein function from the corresponding amino acid sequence. These methods all essentially make use of the “guilty by association” approach. The “guilty by association” approach operates on the general principal that if a given segment of one sequence has a particular property associated with it, then all sequences having that same segment also have that property. The “guilty by association” approach is equally applicable when the subject sequence is a protein sequence. These numerous methods can be divided into a number of well differentiated categories depending on the nature of the exploited information and the manner in which the information is used.

The first category relies on the determination of local or global similarities between a query sequence and annotated sequences in a database. The principle is that if two sequences share one or more regions, then they also share the properties associated with the region, or regions. The validity of this scheme relies on the implicit assumption that two organisms that have extensive genomic similarities also have the same properties. The methods in this category that have been proposed for carrying out protein annotation are numerous.

With similarity-based or homology-based methods there is an inclination by annotators to use either the first or the best match from the output of a database search, i.e., the search carried out by one of the similarity search algorithms, e.g., FASTA, BLAST and Smith-Waterman. However, choosing the first or the best match may not be optimal, especially when dealing with domains that are shared by numerous proteins. For example, the organization of proteins with multiple domains can lead to incorrectly annotated database entries. The use of a domain scan, and the exploitation and analysis of the generated output can substantially improve results. Such a domain scan can be implemented, for example, with the help of the PROSITE, PRINTS, PFAM, BLOCKS or PRODOM databases.

The second category of methods has become known as the “Rosetta stone” approach. With the “Rosetta stone” approach, one seeks to determine groups of proteins that are distinct in a first organism but appear as a single product in a second organism, presumably as a result of a fusion event. Based on this presumption, the distinct proteins in the first organism are assumed to be physically interacting. This comparative information can be helpful in determining the protein properties.

The third category seeks to determine groups of proteins that repeatedly appear close to one another in the chromosomes of different organisms. The proteins of the group that repeatedly appear close to one another are thus assumed to have a functional relationship. Application of this method has found great success with prokaryotic genomes wherein proximal gene organization is manifested in the form of operons. In fact, the method has been used successfully to guide functional annotation. However, it is not evident whether this method applies well to eukaryotic organisms, as eukaryotes lack operons.

A closely related variation of the third category operates on the assumption that if an organism comprises a specific pathway, then the organism will carry all or most of the related genes for that pathway. For example, the work described in “Computational Genetics: Finding Protein Function By Nonhomology Methods,” Curr. Opin. Struct. Biol., 10, 359-65 (2000), the disclosure of which is incorporated by reference herein, attempts to define function in terms of the pathways and complexes in which the protein participates, rather than to suggest a specific biochemical activity. As such, a protein is associated with a function via its linkages to other proteins.

The fourth category seeks to elucidate protein function through analysis of correlated mRNA expression, i.e., the methods commonly implemented in the context of DNA-chip or microarray-chip experiments. The underlying assumption of this fourth category is that functionally related proteins will exhibit correlated mRNA expression levels under multiple experimental settings. The consistent participation of a previously uncharacterized protein in clusters of proteins with understood function, imposes constraints on the possible behavior of the unknown protein within the context of a metabolic pathway.

A more recent variation of this general approach measures the levels of protein expression, rather than the levels of mRNA, with the help of mass spectrometry or two dimensional gel electrophoresis. The method attempts to determine clusters of highly co-expressed proteins. The clusters can then be used to determine the function of any uncharacterized proteins. A detailed description of the methods of protein annotation is provided in I. Rigoutsos et al., “Dictionary-Driven Protein Annotation,” Nucleic Acids Research, vol. 30, no. 17, 3901-16, 2002, the disclosure of which is incorporated by reference herein.

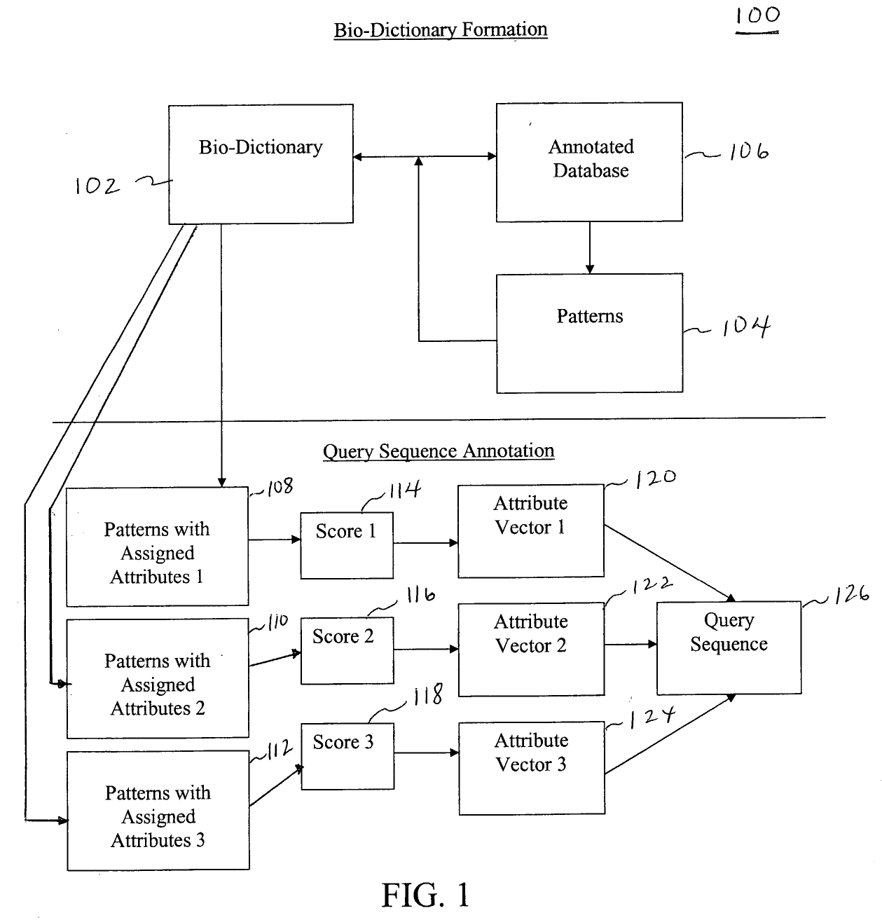

FIG. 1 is a flow chart illustrating an exemplary methodology 100 for annotating a query sequence according to an embodiment of the present invention. The following description of FIG. 1 will first address the formation of a bio-dictionary, and then the annotation of a query sequence. However, while these two main steps of the method may be performed separately, and in the order addressed, the teachings of the present invention should not be construed as being limited to the steps being performed separately or in any prescribed order, and in accordance with the teachings of the present invention, the steps described herein may be performed concurrently.

To form a bio-dictionary 102, patterns 104 associated with annotated database 106 are accessed. Patterns 104 may be derived from annotated database 106. Each pattern of patterns 104, by virtue of the fact that it is a pattern, occurs two or more times in annotated database 106.

The patterns 104 may be assigned attributes based on the annotated sequences of annotated database 106, from which patterns 104 are derived. Patterns with assigned attributes constitute bio-dictionary 102. The attributes represent identified features of the annotated database sequences. Thus, an attribute may represent the following, non-exhaustive list of properties relating to sequences, i.e., annotated database 106: the similarity of a sequence to the sequence, or sequences, of a given known protein; the similarity of a sequence to the sequence, or sequences, representing a given protein family; the likeness of the sequence to all available archaeal, bacterial, eukaryotic and viral sequences, as a function of position within the sequence; the potential secondary structure of the protein encompassing a particular sequence; the cytoplasmic, transmembrane or extracellular behavior of a sequence; the nature and position of binding domains, active sites, post-translationally modified sites and signal peptides; cytoplasmic and extracellular behavior as a function of position within a sequence. A further detailed description of the formation of a bio-dictionary will be presented below.

Annotated database 106 may be any database, or combination of databases, comprising one or more annotated sequences. Annotated database 106 may comprise annotated amino acid sequences encoding the primary structures of proteins. Suitable databases include publicly available databases such as, but not limited to, the SwissProt and the TrEMBL databases. SwissProt is a annotated protein sequence database, and TrEMBL is a computer-annotated supplement of SwissProt (the combination hereinafter referred to as “SwissProt/TrEMBL”).

To annotate a query sequence, patterns with assigned attributes 108, 110 and 112 that match query sequence 126 are selected from bio-dictionary 102. While the present description involves the use of a set number of patterns with assigned attributes, i.e., three patterns with assigned attributes, namely, patterns with assigned attributes 108, 110 and 112, the teachings of the present invention should not be limited to any particular number of patterns or attributes. For example, in accordance with the teachings of the present invention, the number of patterns with assigned attributes may be varied and arbitrary. Each of the patterns with assigned attributes 108, 110 and 112 may be scored. The score can be arbitrarily fixed, or can vary based on a number of predetermined criteria. In an exemplary embodiment a score is used based on a predetermined criteria indicating the degree of similarity between query sequence 126 and the individual sequence, or sequences, of annotated database 106 used to derive patterns 104.

Thus, scores 114, 116 and 118 may be determined for patterns with assigned attributes 108, 110 and 112, respectively. A further detailed description of determining a score will be presented below. Scores 114, 116 and 118 may then be used to determine an amount patterns with assigned attributes 108, 110 and 112 contribute to each of attribute vectors 120, 122 and 124. Attribute vectors 120, 122 and 124 are a representation of the probability that one or more locations within the query sequence 126 contain one or more instances of the particular attributes associated with patterns with assigned attributes 108, 110 and 112. A further detailed description of attribute vectors will be presented below.

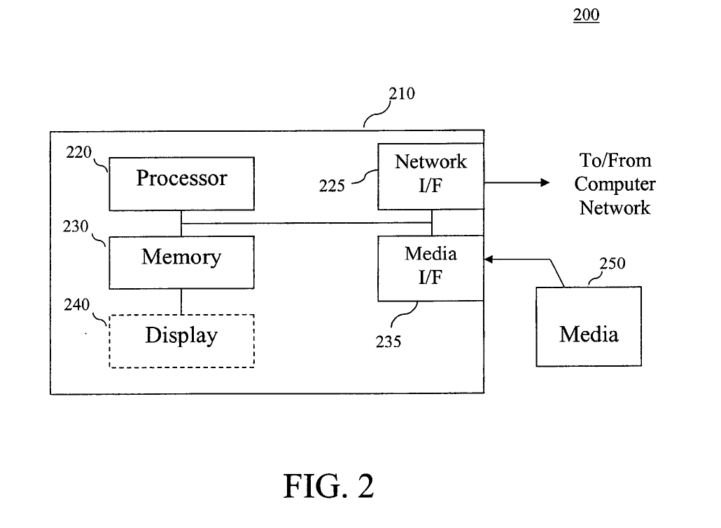

FIG. 2 is a block diagram of an exemplary hardware implementation of a method for annotating a query sequence in accordance with one embodiment of the present invention. It is to be understood that apparatus 200 may implement methodology 100 described above. Apparatus 200 comprises a computer system 210 that interacts with a media 250. Computer system 210 comprises a processor 220, a network interface 225, a memory 230, a media interface 235 and an optional display 240. Network interface 225 allows computer system 210 to connect to a network, while media interface 235 allows computer system 210 to interact with a media 250, such as a Digital Versatile Disk (DVD) or a hard drive.

As is known in the art, the methods and apparatus discussed herein may be distributed as an article of manufacture that itself comprises a machine readable medium containing one or more programs which when executed implement embodiments of the present invention. For instance, the machine readable medium may contain a program configured to access patterns associated with a database comprising annotated sequences; select the accessed patterns that match the query sequence; assign attributes to the patterns based on the annotated sequences; and use the patterns with assigned attributes to analyze the query sequence. The machine readable medium may be a recordable medium (e.g., floppy disks, hard drive, optical disks such as a DVD, or memory cards) or may be a transmission medium (e.g., a network comprising fiber-optics, the world-wide web, cables, or a wireless channel using time-division multiple access, code-division multiple access, or other radio-frequency channel). Any medium known or developed that can store information suitable for use with a computer system may be used.

Processor 220 can be configured to implement the methods, steps, and functions disclosed herein. The memory 230 could be distributed or local and the processor 220 could be distributed or singular. The memory 230 could be implemented as an electrical, magnetic or optical memory, or any combination of these or other types of storage devices. Moreover, the term “memory” should be construed broadly enough to encompass any information able to be read from or written to an address in the addressable space accessed by processor 220. With this definition, information on a network, accessible through network interface 225, is still within memory 230 because the processor 220 can retrieve the information from the network. It should be noted that each distributed processor that makes up processor 220 generally contains its own addressable memory space. It should also be noted that some or all of computer system 210 can be incorporated into an application-specific or general-use integrated circuit.

Optional video display 240 is any type of video display suitable for interacting with a human user of apparatus 200. Generally, video display 240 is a computer monitor or other similar video display.

It is to be understood that the following description exemplifies the formation of a bio-dictionary as referred to in conjunction with the formation of bio-dictionary 102 of FIG. 1. The formation of bio-dictionary 102 involves using a pattern discovery algorithm, such as the Teiresias pattern algorithm, to process very large databases of amino acid sequences and fragments, i.e., annotated database 106, and to derive patterns 104 that appear within individual sequences, as well as within different sequences, i.e., representing different protein families. Patterns such as patterns 104, also referred to as seqlets, have been shown to capture functional and structural properties of the proteins in the databases. Importantly, the patterns, such as patterns 104, may serve to completely describe the sequences of the database at the amino acid level. Following are some examples of patterns with attributes, such as the name of the represented feature or the represented protein family, shown in parentheses:

GDG{IVAMTD}ND{AILV}{PEAS}{AMV} {LMIF}..A (=cation-transporting atpases)

V.I.G.G..G...A (=nad/fad-binding flavoproteins), G..G.GK{ST}TL (=atp/gtp binding P-loop)

KMSKS{LKDIR}{GNDFQ}N (=class I aminoacyl-trna synthetases)

H.....HRD.K..N (=serine/threonine protein kinases)

In terms of the notation used, e.g., {LKDIR} means a choice of exactly one amino acid among the amino acids L, K, D, I and R, written in short form. The symbol ‘.’ denotes a single position wild-card character that can represent any one of the 20 naturally occurring amino acids.

The derived patterns, i.e., patterns 104, may be treated as a current vocabulary for protein sequences to the extent that the database used is kept up to date. The association of patterns 104 with annotation information, which is contained in a typical entry of annotated database 106, comprises bio-dictionary 102. In general, the term bio-dictionary may be used to refer to any collection of patterns. In this particular embodiment, the term bio-dictionary refers to patterns 104 that have been augmented so as to have attributes representing the annotations of annotated database 106 assigned to them.

The key elements behind the bio-dictionary, and details for construction of the bio-dictionary can be found in I. Rigoutsos et al. “Dictionary Building Via Unsupervised Hierarchical Motif Discovery In the Sequence Space of Natural Proteins,” Proteins: Struct. Funct. Genet. 37, 264-77, 1999, the disclosure of which is incorporated by reference herein. The analysis of the three dimensional structural properties associated with the patterns of a bio-dictionary built out of 17 complete archaeal and bacterial genomes are given in I. Rigoutsos et al., “Building Dictionaries of ID and 3D Motifs by Mining the Unaligned ID Sequences of 17 Archaeal and Bacterial Genomes,” Proc. of the Seventh Int. Conf. on Intelligent Systems for Molecular Biology (ISMB '99), the disclosure of which is incorporated by reference herein. A discussion and description of potential uses for the bio-dictionary appear in, I. Rigoutsos, “The Emergence of Pattern Discovery Techniques in Computational Biology,” Metabolic Engineering, 2, 159-77, 2000, the disclosure of which is incorporated by reference herein.

The following is an exemplary methodology for forming bio-dictionary 102. The bio-dictionary 102 should cover, as completely as possible, the sequences of annotated database 106. For the purposes of implementing an embodiment of the present methodology, the May 14, 2001 release of SwissProt/TrEMBL, a large, curated database, serves as a suitable annotated database 106. For example, the May 14, 2001 release comprises 532,621 amino acid sequences and fragments with a grand total of 170,762,058 amino acids.

The May 14, 2001 release of the SwissProt/TrEMBL database may be processed in two phases. In the first phase, the Teiresias algorithm (using the parameters L equals eight, W equals eight and K equals two) generates variable length patterns containing no wild cards. L and W represent integers defining the density of a pattern. K represents the minimum number of patterns within parameters L and W. The density of a pattern may be described as the minimum amount of homology between any two sequences of a group, the group consisting of all sequences obtained from a certain pattern by replacing all wild card positions with one of the 20 amino acids. Thus, a pattern has an<L, W>density if every substring of the pattern that starts and ends with an amino acid and has a minimal length W and contains L or more amino acid residues. The use of the Teiresias algorithm to derive patterns is described in U.S. patent application Ser. No. 09/582,044, filed Jun. 21, 2000, entitled “Method and Apparatus for Performing Sequence Homology Detection,” the disclosure of which is incorporated by reference herein.

In the second phase, all instances of the patterns in the database may be located and masked, except for the one pattern that appears in the longest database sequence. The Teiresias algorithm may then be rerun on the database sequences corresponding to the masked patterns, but this time using L equals six and W equals 15. The exemplary processing described herein would require approximately 45 CPU days worth of computation using IBM RS64III processors with a clock speed of 450 MHz. The use of a parallel implementation of Teiresias developed for shared memory architectures, may help completion of this computation in about two days on a 24-processor IBM S-80 supercomputer.

The two pattern discovery phases generate a bio-dictionary suitable for use in the present invention. The exemplary bio-dictionary, as described herein, would contain a combined total of 42,996,454 patterns accounting for 98.2 percent of the database sequences at the amino acid level. The length of each pattern is approximately 12 to 13 amino acids. According to the methods highlighted above, the exemplary bio-dictionary will likely contain redundant patterns, i.e., a given amino acid position in the processed database would participate in, and be covered by, multiple patterns. The redundancy of representation is a desired property to be exploited during annotation. The methodology for creating a bio-dictionary is described in U.S. patent application Ser. No. 09/582,045, filed Jun. 21, 2000, entitled “Method and Apparatus for Performing Pattern Dictionary Formation For Use in Sequence Homology Detection,” the disclosure of which is incorporated by reference herein

As described above, the annotations of annotated database 106 are used to assign attributes to patterns 104. Any information, or category of information, of any database would be suitable for assigning attributes to the patterns in accordance with the teachings of the present invention. For example, a suitable database is the Protein Databank (PDB). The PDB contains protein structures. Patterns may be associated with the three-dimensional structures in the database and sequence annotation may be conducted in accordance with the present invention.

The annotation information contained in annotated database 106 may be derived from predetermined entries, or categories of entries. In an exemplary embodiment, the SwissProt/TrEMBL database is used. The SwissProt/TrEMBL database comprises a plurality of line code categories, each line code category providing a distinct body of information. For example, the ID line, or identification line provides information including the protein name. Another line code category is the OC line, or the organism classification line. The OC line provides taxonomic classification information for the source organism. A further line code category is the FT line, or feature table line. The FT line highlights regions or sites of interest in the sequence. The FT line contains information about features of a sequence followed by numbers corresponding to the amino acid residues that mark the endpoints, i.e., extent, of the feature in the sequence. The FT line ends with additional information about the features. The following is a partial list of FT line labels present in the SwissProt/TrEMBL database, that are used in the exemplary analysis described herein:

| | | | | |
|---|---|---|---|---|
| Mod_res | lipid | disulfid | thioeth | thiolest |
| Carbohyd | metal | binding | transit | signal |
| Propep | chain | peptide | ca_bind | domain |
| Dna_bind | np_bind | transmem | zn_fing | similar |
| Act_site | site | init_met | non_cons | non_ter |
| Helix | strand | turn | se_cys | |

Attributes are derived from the information contained in the predetermined line code categories.

It is to be understood that the following description exemplifies sequence annotation as referred to in conjunction with the annotation of query sequence 126 of FIG. 1. When presented with a query sequence to annotate, the following illustrative operations may be performed:

1) determine the subset S of seqlets in the Bio-Dictionary that match regions

in the query Q with length |Q| ;

2) for each seqlet s in S do {
2a) let q_{from} and qₜₒ denote the region in the query matched by s ;
2b) use the Bio-Dictionary information to access all instances of seqlet s

in the SwissProt/TrEMBL database and let P denote the set of
corresponding SwissProt/TrEMBL entries ;

2c) for each SwissProt/TrEMBL entry p in P {

- let {p_{from}, pₜₒ} denote the instance of seqlet s in the SwissProt/TrEMBL
entry p under consideration ;
- retrieve full SwissProt/TrEMBL record R for the respective entry p ;

- retrieve organism classification OCₚ from the record R for p ;
- if (OCₚ has not been encountered before) {

- create a one-dimensional score array with length |Q| ;
- initialize the array to all 0's and set OCₚ as its attribute ;
- assign CONTRIB({p_{from}, pₜₒ}, s) to the interval {q_{from}, qₜₒ} of

this new array ;

}

else {

- add CONTRIB({p_{from}, pₜₒ}, s) to interval {q_{from}, qₜₒ} of the already

existing array with attribute Ocₚ ;

}

- retrieve description DEₚ from the record R for p ;
- if (DEₚ has not been encountered before) {

- create a one-dimensional score array with length |Q| ;
- initialize the array to all 0's and set DEₚ as its attribute ;
- assign CONTRIB({p_{from}, pₜₒ}, s) to the interval {q_{from}, qₜₒ} of

this new array ;

}

else {

- add CONTRIB({p_{from}, pₜₒ}, s) to interval {q_{from}, qₜₒ} of the already

existing array with attribute DEₚ ;

}

- from the record R, retrieve all features FTₚ that overlap with the

instance {p_{from}, pₜₒ} of s in the containing sequence ;

- determine the interval of intersection {i_{from}, iₜₒ} of each annotated

region in R with the instance {p_{from}, pₜₒ} of s ;

- for each feature f in FTₚ with non-zero intersection {i_{from}, iₜₒ} {

if (f has not been encountered before) {

- create a one-dimensional score array with length |Q| ;
- initialize the array to all 0's and set f as its attribute ;
- assign CONTRIB({p_{from}, pₜₒ}, s) to the interval

{q_{from}+ (i_{from}−p_{from}), q_{from}+ (iₜₒ−p_{from})} of this new array;

}

else {

- add CONTRIB({p_{from}, pₜₒ}, s) to the interval

{q_{from}+ (i_{from}−p_{from}), q_{from}+ (iₜₒ−p_{from})} of the already existing
array with attribute f ;

}

}

2d) OPTIONAL STEP - repeat this process for other useful information in record R
;
}

Patterns with assigned attributes 108, 110 and 112 are then compared to query sequence 126. Any one of patterns with assigned attributes 108, 110 or 112 may have more than one attribute assigned to it. If the pattern under consideration has an attribute attached to it that has not yet been encountered in relation to the particular query sequence, then an attribute vector for that new particular attribute, is created. It is to be understood that the present description exemplifies the defining of an attribute vector as referred to in conjunction with the defining of attribute vectors 120, 122 and 124 of FIG. 1. Additionally, for ease of reference, the defining of an attribute vector will be described before the determining of a score for the patterns is described. An attribute vector is a convenient representation of information about the presence of a particular attribute in the query sequence. The attribute vector described herein may contain a number of place holders equal to the length of the query sequence. However, while the present description involves use of an attribute vector with place holders, any vector structure would be suitable in accordance with the teachings of the present invention. Further, any other data structure that permits the storage and access of information relating to annotation information may be used in the present invention.

Each of the place holders in the attribute vector is associated with an accumulator, i.e., a counter. The counter initially has a value of zero. The pattern contributes to a region {q_{from}, qₜₒ} of the attribute vector by contributing a value to the counters that correspond to the region, or regions, {q_{from}, qₜₒ} of the query sequence that are matched by the pattern. The counter, or counters, that have a value contributed to them are denoted by indicating the beginning and ending units, i.e., {q_{from}, qₜₒ} of the region. Thus, the first unit to the fifth unit would be presented as {1, 5}. The pattern may contribute values to the attribute vector in the form:

CONTRIB({p_{from},pₜₒ},s)

wherein the above expression indicates the amount of contribution a particular pattern, in this case pattern s, has contributed to the attribute vector in the region {q_{from}, qₜₒ}. The query sequence is thus annotated incrementally, one pattern at a time, by reference to the attributes of the matching pattern, or patterns, the patterns in turn being derived from the annotated database sequences.

If, on the other hand, a pattern has an assigned attribute that has already been encountered, the pattern merely adds the corresponding contribution value to the already existing value, or values of the corresponding counter, or counters. In the situation wherein the attribute has already been encountered and an attribute vector for that attribute already exists, additional patterns may contribute to the same counter, or counters, {q_{from}, qₜₒ} as previous patterns, or to different counters {q′_{from}, q′ₜₒ}, depending on which counter each pattern matches. Thus, the units {q_{from}, qₜₒ} to which the patterns contribute may or may not be overlapping.

After all patterns in the bio-dictionary have been exhausted, the attribute vectors may be sorted and ranked based on the total amount of accumulated contributions each attribute vector receives from the patterns. Any other suitable ranking or sorting methodologies may be used in accordance with the teachings of the present invention. The attribute vectors may be grouped into categories, i.e., by attribute, and ranked separately within each category. The top ranking vectors, T, of each category may be identified, to be presented to a user of the methodology in a coherent order. Each of these attribute vectors will contain non-zero values at precisely those counters {q_{from}, qₜₒ} that were matched by patterns carrying the same attribute.

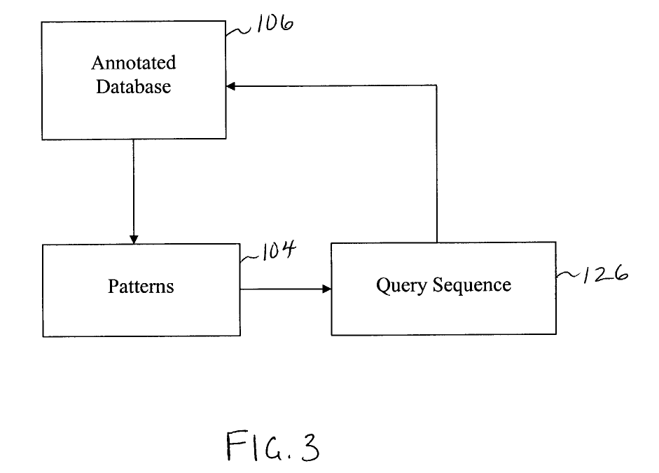

The annotation of the query sequence and the association of patterns with the corresponding information from the annotated sequences of the annotated database 106 may be performed in any order. For example, as is shown in FIG. 1, attributes are first assigned to patterns 104 to form the patterns with assigned attributes comprising bio-dictionary 102, and then patterns with assigned attributes 108, 110 and 112 are used to annotate query sequence 126. Alternatively, as shown in FIG. 3, annotated database 106, as also shown in FIG. 1, comprising annotated sequences is used to derive patterns 104, as also shown in FIG. 1. Patterns 104 are then compared with the query sequence 126, as also shown in FIG. 1. Attributes are then assigned to the patterns 104 that match query sequence 126 using annotated database 106.

Generally, the bio-dictionary formed should not be seen as a collection of patterns each of which necessarily captures a single, unique attribute of the database sequence, such as a kinase domain or a metal binding site. While patterns assigned a specific, single attribute may be used in accordance with the teachings of the present invention, by design many of the patterns may also carry multiple attributes. A pattern can match multiple regions of the database sequences, the regions crossing functional and structural boundaries. As such, these patterns may be assigned multiple attributes. The patterns being assigned multiple attributes is different than the one-to-one correspondence typical of predicate-containing databases such as PROSITE, PRINTS or INTERPRO.

Similarly, the bio-dictionary may also contain multiple patterns all of which are assigned the same attribute, or attributes. Further, there may be patterns that overlap with one another. Thus, a given region of a query sequence may also be covered by multiple patterns. Each of the patterns covering a region of the query sequence will in general be assigned one or more attributes that are used to analyze the query sequence by coloring the corresponding region, or regions, of the query sequence. When multiple patterns match a particular region of the query sequence, the patterns and the respective assigned attributes, may be ranked. For example, let a given region of the query sequence match a number of distinct patterns, M. In order for an attribute, e.g., a metal binding site, to gain a high ranking in the reported results, a large portion of M patterns must be assigned this attribute.

By definition, each of the patterns of the bio-dictionary must represent at least two regions in the database 106. Thus, if M patterns cover a given region in the query sequence, then the following two properties will simultaneously hold:

there exists the total of the database sequences, F, corresponding to all of the instances of the patterns, M, in the database, the database sequences, F, being similar with the amino acid neighborhood surrounding this query position; and

the database sequences, F, will concur on the identity of each amino acid contained in each of the patterns, M.

The database sequences, F, however, may or may not concur on the attribute to annotate the particular region of the query sequence. If N number of the F database sequences have a particular attribute, i.e., a metal binding site, at a particular region, then by the “guilty by association” approach, the chance that the same region of the query sequence also has that attribute, i.e., is also a metal binding site, will be proportional to N/F. This concept may be applied to every attribute that is attached to a pattern.

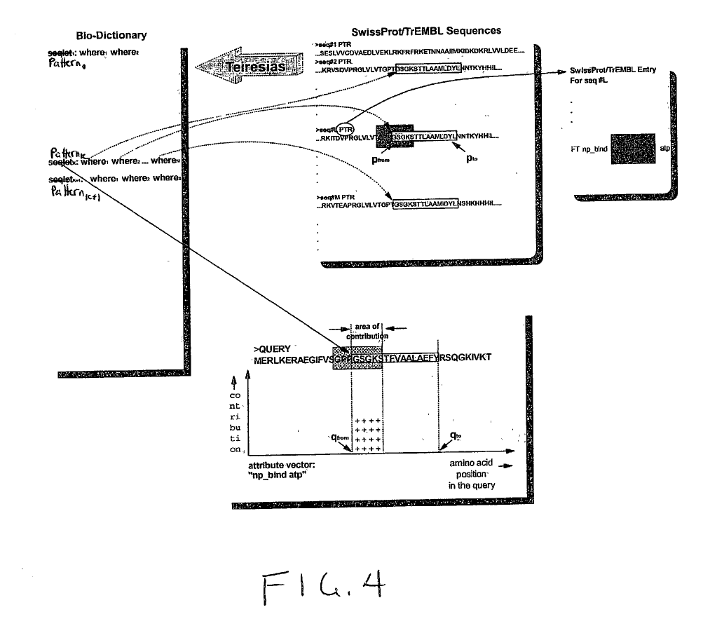

FIG. 4 is a schematic diagram illustrating an exemplary implementation of the present invention. As is shown in FIG. 4, a pattern does not have to match an entire region of a database sequence, or sequences, to be useful in analyzing a query sequence. Further, FIG. 4 shows that a pattern also does not have to have an attribute explicitly linked with it to be useful in analyzing the query sequence, as shown in conjunction with sequence #2 and sequence #M in the SwissProt/TrEMBL database. In FIG. 4 it is shown that a query sequence is annotated using a bio-dictionary, and that pattern_{K} matches the region {q_{from}, qₜₒ} in the query sequence. During the formation of the bio-dictionary it was determined that pattern_{K} matches three regions in the SwissProt/TrEMBL database. Following these three regions back to the database entries, it can be determined that in one of the database sequences, pattern_{K} spans an interval, {q_{from}, qₜₒ}, of a region of the database sequence, {feat_{from}, featₜₒ}, that is annotated as “np_bind atp,” i.e., as atp-binding. The interval {i_{from}, iₜₒ} denotes the intersection of the intervals {p_{from}, pₜₒ} and {feat_{from},featₜₒ}. In this particular example, pattern_{K} contributes to the hypothesis of the presence of a partial atp-binding domain in the query sequence by incrementing the support at the locations {q_{from}+(i_{from}−p_{from}), q_{from}+(iₜₒ−p_{from})} of the “np_bind atp” attribute vector, shown as the area of contribution.

If the query sequence contains a given attribute, then each one of the potentially numerous patterns that match the region of the query sequence corresponding to the attribute will cumulatively, as well as independently, provide support for the attribute at the respective region. Conversely, the number of patterns matching the query sequence may be used to determine whether the query sequence actually contains a given attribute. Namely, as the accumulated support for the attribute increases, i.e., as the number of patterns with the assigned attribute that match the region increases, so does the likelihood of the presence of the attribute in the query sequence.

An attribute vector may be defined from the patterns with assigned attributes, the attribute vector representing the query sequence, as described in conjunction with the defining of attribute vectors 120, 122 and 124 of FIG. 1. Following from the description of query sequence annotation above, if the query sequence is a true member of a known protein family, then it is expected that the attribute vector for this family will obtain support along its length from each pattern that matches the query sequence. Similarly, if a query sequence comprises a global region, i.e., domain, that is well represented in the database sequences, then it is likely that the attribute vector for the query sequence will have values corresponding to that region of the query sequence. In an analogous manner, if the query sequence shares only a local region with the same domain, then the corresponding attribute vector will have non-zero values corresponding only to the query sequence region overlapping the domain.

The situation may arise wherein the query sequence contains only a portion of a region from a database sequence, or sequences, i.e., a query sequence with only the first 20 amino acids of a protein kinase domain. In this situation it is helpful to further calculate the minimum, average and standard deviation values for the expected size of each of the T top ranking attributes as this can be determined by the contents of database 106. This permits one to easily determine whether the query sequence represents a complete instance of the stated attribute or only a fragment.

In the context of protein sequence annotation, the present invention allow for the determination of the following, non-exhaustive list of properties, that includes but is not limited to: local and global similarities between the query sequence and any protein already present in any available database; the likeness of the query sequence to all available archaeal, bacterial, eukaryotic and viral sequences in a database as a function of amino acid position within the query sequence; the character of the secondary structure of the query sequence as a function of amino acid position within the query sequence; the cytoplasmic, transmembrane or extracellular behavior of the query; the nature and position of binding domains, active sites, post-translationally modified sites and signal peptides; cytoplasmic and extracellular behavior; and the similarity of the query sequence to each of the three phylogenetic domains as a function of amino acid position.

It is to be understood that the following description exemplifies the determining of a score for the patterns with assigned attributes, as referred to in conjunction with the determining of scores 114, 116 and 118 for patterns with assigned attributes 108, 110 and 112 of FIG. 1. In accordance with the teachings of the present invention, a weighted, position-specific scoring scheme may be used. The weighted, position-specific scoring scheme of the present invention is unaffected by the overrepresentation in the database of well conserved proteins and protein regions.

Above, it was described how the patterns with assigned attributes are used to contribute values to counters of the attribute vector corresponding to portions of the query sequence matched by the patterns. The amount each pattern will contribute to counters of the attribute vector corresponding to portions of the query sequence matched by the patterns will now be described.

For example, if pattern_{K} is one of the patterns matching a region of the query sequence, then qᵢ_{₁}qᵢ_{₂}qᵢ_{₃} . . . qᵢ_{ₗ} and Pⱼ_{₁}pⱼ_{₂}pⱼ_{₃} . . . pⱼ_{ₗ} may be used to denote the amino acid sequences representing instances of pattern_{K} in the query sequence and in the database sequence, d, respectively. Further, {i₁, . . . iₗ} and {j₁, . . . jₗ} may be used to denote the endpoints of the regions spanned by the pattern in the query sequence and the database sequence, d, respectively. Further, any pattern, i.e., pattern_{K}, that matches an entire region of database sequence, d, annotated with attribute A, is also annotated with attribute A.

Exemplary pattern_{K} may also bring together two sequence fragments each with lengths, i.e., measured as the number of amino acids in the sequence, equal to the span of the pattern_{K}, one fragment coming from the query sequence and the other coming from the database sequence d. The more similar these two fragments are to each other, the more likely it is that upon completion of the annotation of the query sequence, the attribute A that is associated with the region of database sequence, d, pⱼ_{₁}pⱼ_{₂}pⱼ_{₃} . . . pⱼ_{ₗ} will be carried over to the region of the query sequence qᵢ_{₁}qᵢ_{₂}qᵢ_{₃} . . . qᵢ_{ₗ} through the “guilty by association” approach. There is a rather straightforward manner in which pattern_{K} can contribute to the attribute vector for attribute A. A scoring matrix is used to generate contributions in a position- and content-dependent manner as follows:

for m=1 to l{attribute_vector {i₁+m−1}=attribute_vector+f(scoring_matrix[qᵢ_{₁}₊ₘ₋₁][pⱼ_{₁}₊ₘ₋₁])}

wherein m is a variable equivalent to the endpoints i of the region spanned by the pattern in the query sequence and j of the region spanned pattern in the database. In other words, the pattern will contribute to the (i₁+m−1)-th unit of the attribute vector an amount that relates to the degree of similarity between the amino acids occupying the positions qᵢ_{₁}₊ₘ₋₁ and pⱼ_{₁}₊ₘ₋₁ respectively. Function f (.), above, may be f(x)=2^{x}+const. The scoring matrix, scoring_matrix, used can be any of the standard PAM or BLOSUM scoring matrices.

In order to avoid the effects of a given protein family or fragment being over represented in the, i.e., SwissProt/TrEMBL, database, the additional constraint may be imposed that a given pattern cannot contribute to the same attribute vector more than once. In other words, if exemplary pattern_{K} captures a well conserved region that thus appears in a large number of SwissProt/TrEMBL database sequences, only one instance of the pattern will contribute to the respective attribute vector.

A given pattern with assigned attributes will contribute to each of the attribute vectors that correspond to those attributes. The amount of these contributions will depend on how well an annotated database sequence with an instance of the attribute matches the instance in the query sequence. Thus, different attribute vectors will accumulate different amounts of contribution from the different patterns. Further, the amounts of these contributions will also depend on the position within the attribute vector.

During the annotation of the query sequence, a bookkeeping array, total, is maintained representing a sequence of a length equal to that of the query sequence. For every pattern with amino acid sequences representing an instance qᵢ_{₁}qᵢ_{₂}qᵢ_{₃} . . . qᵢ_{ₗ} in the query sequence, total is updated as follows:

for m=1 to l{total{i₁+m−1}=total{i₁+m−1}+f(scoring_matrix[qᵢ_{₁}₊ₘ₋₁][pⱼ_{₁}₊ₘ₋1])}

Thus, the i-th position of total is a number representing the number of patterns that have contributed to it. Each contribution is weighted by the degree of similarity between the amino acids in the query sequence and the corresponding database sequence, as is done in defining the attribute vector. The function f (.) above, may be f(x)=2^{X}+const. Note that at all times during processing, the value of total {i} is greater than or equal to the maximum value encountered in the i-th position of any of the attribute vectors for this query sequence.

Once all of the patterns matching the query sequence have been examined, the contents of the i-th position of each attribute vector are normalized by dividing by the value of total {i}. Multiplying the normalized value by 100 gives, for each attribute vector, a measure of the fraction of the total contribution that this attribute vector has received, as a function of position within the query sequence. Well conserved attributes are matched by a greater number of patterns, and thus will receive values close to 100 percent. Less well conserved attributes will be matched by fewer patterns and thus will receive lesser values. This particular way of normalizing additionally prevents the situation wherein regions of the query sequence having equal lengths receive disproportionately different contributions due to differences in the number of contributing patterns, i.e., as a result of overrepresentation in the database.

Once the units of the attribute vectors have been normalized, the units are sorted based on the total amount of contributions received. The top, T, ranking vectors are noted. Finally, an additional requirement may be imposed that any reported attributes be supported by non-zero values over a minimum number X of counters, the value of X being user-defined.

Although illustrative embodiments of the present invention have been described herein, it is to be understood that the invention is not limited to those precise embodiments, and that various other changes and modifications may be effected therein by one skilled in the art without departing from the scope or spirit of the invention. The following examples are provided to illustrate the scope and spirit of the present invention. Because these examples are given for illustrative purposes only, the invention embodied therein should not be limited thereto.

### EXAMPLES

In the following examples, a carefully selected collection of example query sequences are annotated using the teachings of the present invention.

### Example 1

### UBIQ_HUMAN

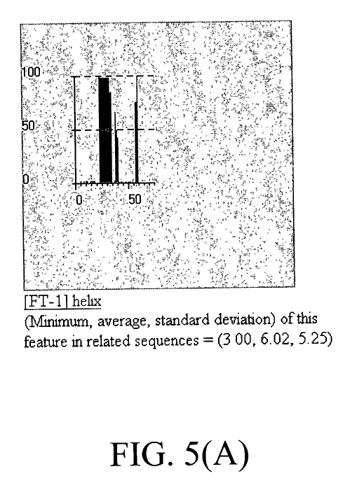

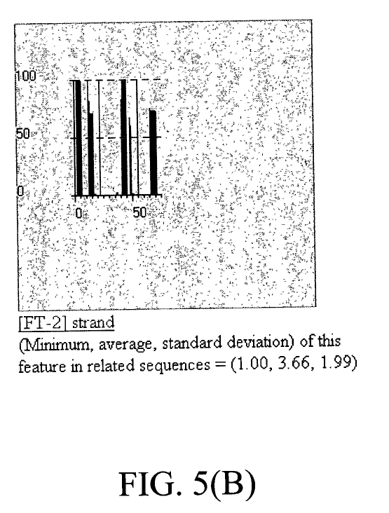

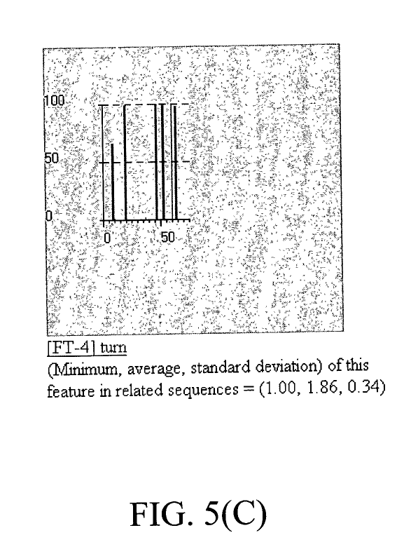

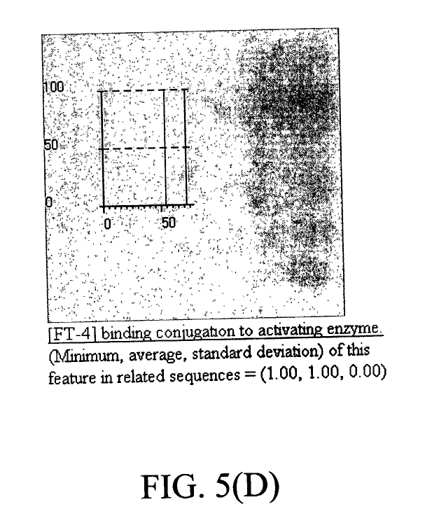

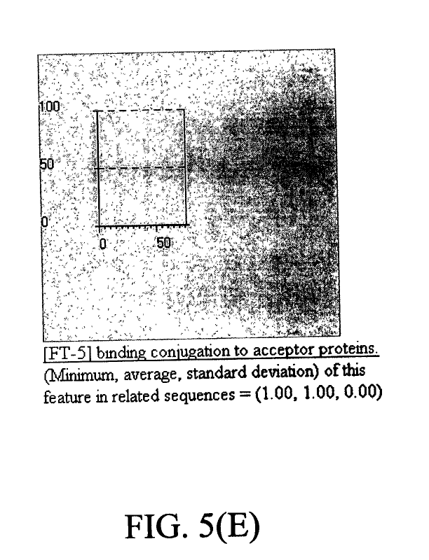

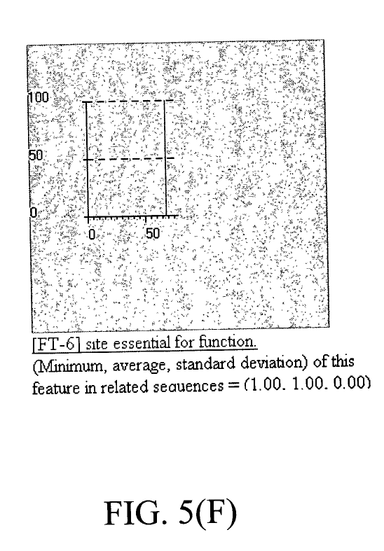

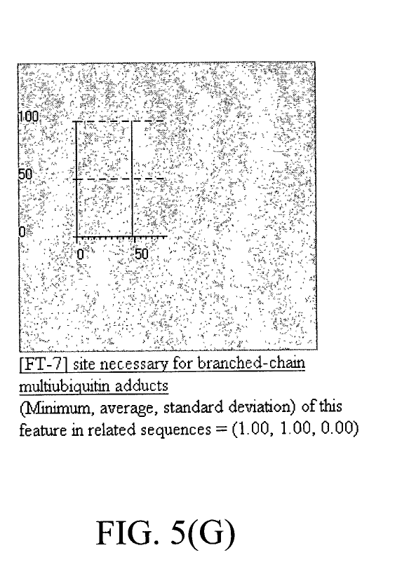

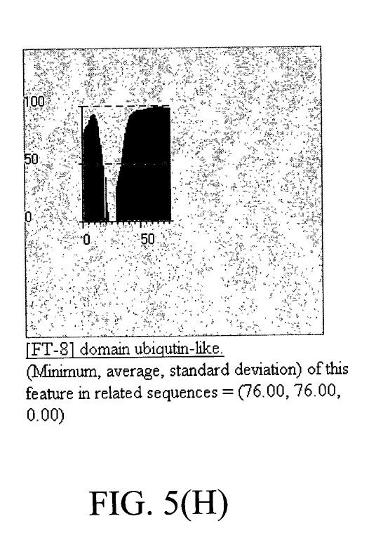

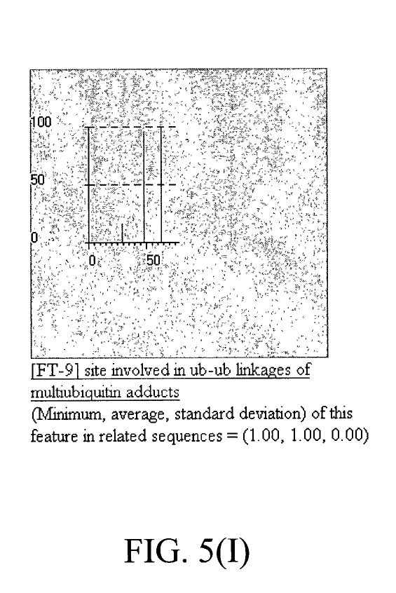

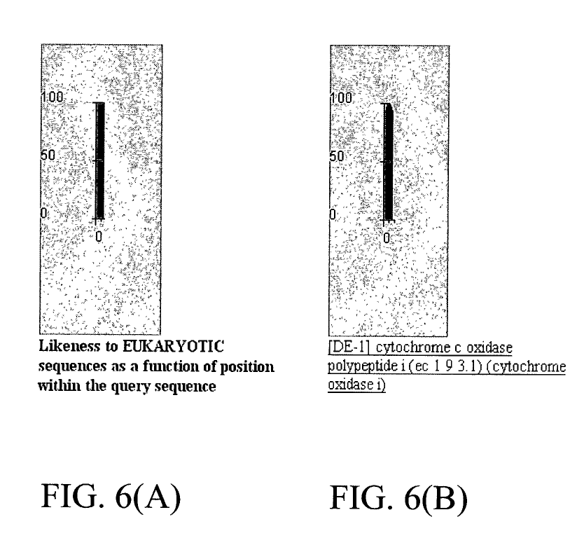

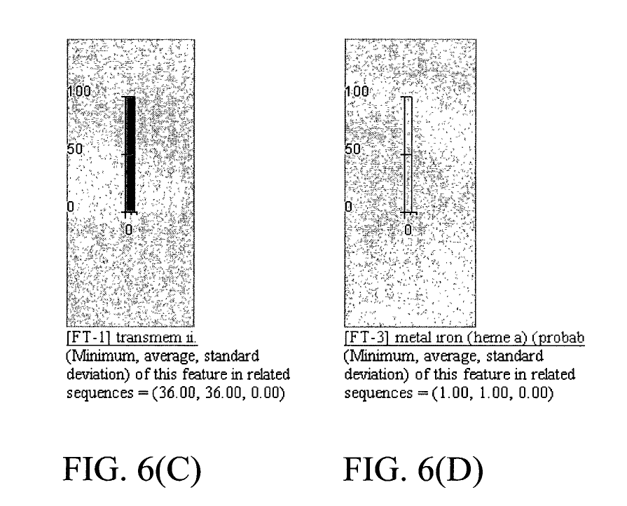

The first example examines the annotation of the 76 amino acid query sequence representing human ubiquitin, UBIQ_HUMAN. The results of the analysis are shown in FIG. 4, FIG. 5 and FIG. 6. As can be seen from FIG. 4, FIG. 5 and FIG. 6, the SwissProt/TrEMBL database contains enough information for our method to correctly determine the secondary structure of the fragment. The localization and interweaving of the helices, strands and turns may be seen in FIG. 4. It is important to note how the method correctly determines the nature and position of seven sites that are relevant to the function of ubiquitin, as well as the presence and extent of the ubiquitin domain.

### Example 2

### A Very Short Fragment

The second example involves the eight amino acid fragment VVVTAHAF, a fragment that is too short to be used with heuristics-based similarity search algorithms such as FASTA and BLAST/PSI-BLAST. As shown in FIGS. 6(A)-(D), processing of the fragment with the present methodology allows for the determinations that:

a) the fragment is an amino acid combination encountered only in the eukaryotic domain;

b) the fragment belongs to a cytochrome-c oxidase;

c) the fragment is part of a transmembrane domain; and

d) the fragment has a metal (iron) binding site at the sixth amino acid position, i.e., H (histidine).

### Example 3

### ACTR_BOVIN

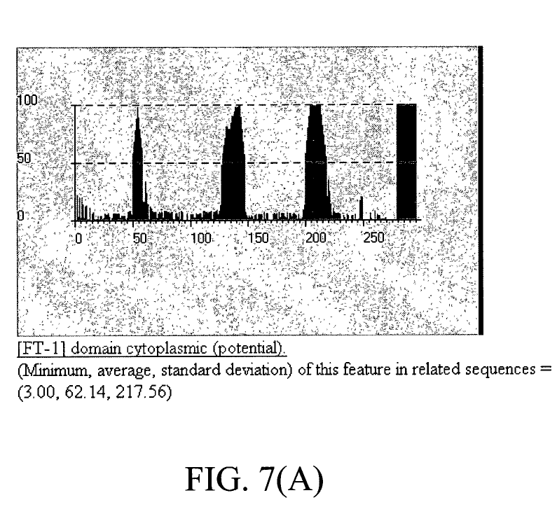

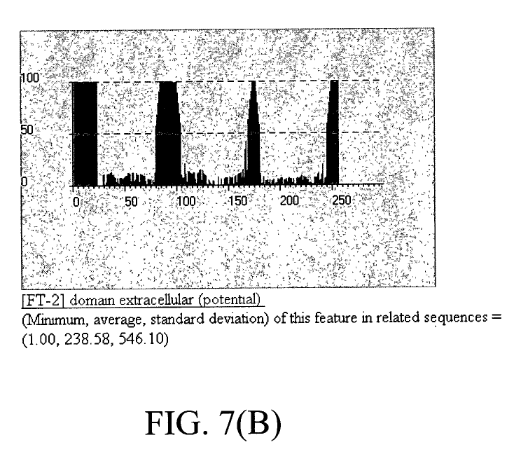

The methodology of the present invention may be further used to determine cytoplasmic, transmembrane and extracellular regions in a given query sequence. In this example, ACTR_BOVIN, an adrenocorticotropic hormone receptor protein from B. Taurus is used as an exemplary query sequence. FIGS. 7(A)-(B) show plots for the cytoplasmic and extracellular behavior of the query sequence. The regions of the query sequence that are not accounted for by these two plots correspond precisely to the seven transmembrane domains of the ACTR_BOVIN (which are not shown).

What is claimed is:

1. A method for annotating a query sequence, the method comprising the steps of:
accessing patterns associated with a database comprising annotated sequences;
assigning attributes to the patterns based on the annotated sequences; and
using the patterns with assigned attributes to analyze the query sequence.

2. The method of claim 1, further comprising the step of selecting the accessed patterns that match the query sequence.

3. The method of claim 1, further comprising the step of storing the patterns with assigned attributes in a database.

4. The method of claim 1, wherein the using step further comprises the step of defining an attribute vector from the patterns with assigned attributes, the attribute vector characterizing portions of the query sequence.

5. The method of claim 1, wherein the query sequence is a polypeptide sequence comprising amino acid residues.

6. The method of claim 4, wherein the attribute vector comprises a number of counters.

7. The method of claim 6, wherein the query sequence is a polypeptide sequence comprising amino acid residues and the number of counters is proportional to the number of amino acid residues in the query sequence.

8. The method of claim 6, wherein the assigned attributes are used to contribute values to counters of the attribute vector corresponding to portions of the query sequence matched by the patterns.

9. The method of claim 4, comprising a plurality of attribute vectors.

10. The method of claim 9, wherein the values contributed to the counters of each of the attribute vectors of the plurality of attribute vectors are normalized.

11. The method of claim 9, wherein each attribute vector of the plurality of attribute vectors represents a different attribute.

12. The method of claim 9, wherein the plurality of attribute vectors are ranked.

13. The method of claim 12, wherein the top ranking attribute vectors are reported.

14. The method of claim 1, further comprising the step of determining a score for the patterns with assigned attributes used to contribute to the attribute vector.

15. The method of claim 14, wherein the score represents a degree of similarity between the query sequence and the annotated sequences of the database.

16. The method of claim 15, wherein the score is normalized.

17. The method of claim 1, wherein the attributes relate to at least one of secondary structure characteristics of the query, presence of known domains, signal peptides, active sites, post-translationally modified sites, cytoplasmic behavior, extracellular behavior, and similarity of the query to each of the three phylogenetic domains as a function of amino acid position.

18. An apparatus for annotating a query sequence, the apparatus comprising:
a memory; and
at least one processor, coupled to the memory, operative to:
access patterns associated with a database comprising annotated sequences;
assign attributes to the patterns based on the annotated sequences; and
use the patterns with assigned attributes to analyze the query sequence.

19. The apparatus of claim 18, wherein the at least one processor is further operative to select the accessed patterns that match the query sequence.

20. The apparatus of claim 18, wherein in accordance with the using operation the at least one processor is further operative to define an attribute vector from the patterns with assigned attributes, the attribute vector characterizing portions of the query sequence.

21. The apparatus of claim 18, wherein the query sequence is a polypeptide sequence comprising amino acid residues.

22. The apparatus of claim 20, wherein the attribute vector comprises a number of counters.

23. The apparatus of claim 22, wherein the query sequence is a polypeptide sequence comprising amino acid residues and the number of counters is proportional to the number of amino acid residues in the query sequence.

24. The apparatus of claim 22, wherein the assigned attributes are used to attach meanings to counters of the attribute vector corresponding to portions of the query sequence matched by the patterns.

25. The apparatus of claim 18, wherein the at least one processor is further operative to determine a score for the patterns with assigned attributes used to define the attribute vector, wherein the score represents a degree of similarity between the query sequence and the annotated sequences of the database.

26. An article of manufacture for annotating a query sequence, comprising a machine readable medium containing one or more programs which when executed implement the steps of:
accessing patterns associated with a database comprising annotated sequences;
assigning attributes to the patterns based on the annotated sequences; and
using the patterns with assigned attributes to analyze the query sequence.

27. The article of manufacture of claim 26, further comprising the step of selecting the accessed patterns that match the query sequence.

28. The article of manufacture of claim 26, wherein the using step further comprises defining an attribute vector from the patterns with assigned attributes, the attribute vector characterizing portions of the query sequence.

29. The article of manufacture of claim 26, wherein the query sequence is a polypeptide sequence comprising amino acid residues.

30. The article of manufacture of claim 28, wherein the attribute vector comprises a number of counters.

31. The article of manufacture of claim 30, wherein the query is a polypeptide sequence comprising amino acid residues and the number of counters is proportional to the number of amino acid residues in the query sequence.

32. The article of manufacture of claim 30, wherein the assigned attributes are used to attach meanings to counters of the attribute vector corresponding to portions of the query sequence matched by the patterns.

33. The article of manufacture of claim 26, further comprising the step of determining a score for the patterns with assigned attributes used to define the attribute vector, wherein the score represents a degree of similarity between the query sequence and the annotated sequences of the database.
